(19) United States
(12) Patent Application Publication
(10) Pub. No.: US 2012/0035701 A1
(43) Pub. Date: Feb. 9, 2012

(54) STENT STRUT APPOSITIONER

(76) Inventor: JOHN T. TO, Newark, CA (US)

(21) Appl. No.: 13/209,370

(22) Filed: Aug. 13, 2011

Related U.S. Application Data
(63) Continuation-in-part of application No. PCT/US2010/024601, filed on Feb. 18, 2010.
(60) Provisional application No. 61/154,440, filed on Feb. 23, 2009.

(51) Int. Cl. A61F 2/84

(52) U.S. Cl. 623/1.11

(57) ABSTRACT

The teachings provided herein are generally directed to a method, device and system for use in reducing, inhibiting, or correcting a malapposed intraluminal device. An example of such an intraluminal device is a stent and, in some embodiments, the intraluminal device can be any device known to one of skill, the implementation of which might benefit from the teachings provided herein. In some embodiments, the intraluminal device is a cardiac stent.

### CROSS-REFERENCE TO RELATED APPLICATIONS

This application is a continuation-in-part of International Patent Application No. PCT/US2010/024601, filed Feb. 18, 2010, which claims the benefit of U.S. Provisional Application No. 61/154,440, filed Feb. 23, 2009; each of which is hereby incorporated herein by reference in its entirety.

### BACKGROUND

1. Field of the Teachings

The teachings are directed to a method, device and system for use in reducing, inhibiting, or correcting a malapposed intraluminal device.

2. Description of the Related Art

Intraluminal medical devices are inserted at a target location in a lumen of a subject, and are often intended to contact the lumen surface during placement. A poor contact between the device and the lumen surface can create adverse consequences.

stent is a device that holds tissue in place and is often used to support tissues while healing takes place. A stent can keep “tube-shaped” structures such as, for example, blood vessels, open after surgery. Stents may also be used, for example, in creating an arterio-venous fistula in a hemodialysis patient, reattaching intestines after a temporary colostomy, and keeping a ureter open after surgical removal of a blockage in the ureter. Typically, stents can be compressed to a reduced diameter, inserted through small lumens with catheters and expanded to a larger diameter when positioned at a desired location. Examples of stents in the patent literature include U.S. Pat. Nos. 4,733,665, 4,800,882, and 4,886,062.

In the early 1990's, physicians began using stents to improve angioplasty procedures. An intraluminal coronary artery stent can be a small, self-expanding or expandable, stainless steel mesh tube that is placed within a coronary artery to keep a grafted vessel open during a coronary artery bypass graft surgery or after balloon angioplasty to prevent restenosis of the vessel.

In a typical percutaneous transluminal coronary angioplasty (PTCA) procedure, a guiding catheter is inserted percutaneously through a brachial or femoral artery and advanced through the vasculature until the distal end of the guiding catheter is in a desired position within a lumen of a coronary artery. A guidewire and a dilatation catheter having a balloon on the distal end are introduced through the guiding catheter. The guidewire is advanced into the patient's coronary vasculature, and the dilatation catheter is advanced over the guide wire until the balloon is properly positioned in the lumen of the artery across an arterial lesion such as, for example, a lesion of atherosclerotic plaque or a calcified lesion.

The balloon is inflated to a predetermined size with a radiopaque liquid at a relatively high pressure to radially compress the lesion and remodel the lumen of the artery. The balloon is deflated, the dilatation catheter is withdrawn from the patient, and the blood flow is resumed through the dilated artery. To avoid restenosis, an intravascular stent can be implanted to maintain vascular patency. The stent is typically transported through the patient's vasculature on the balloon portion of a catheter.

A problem existing in the art is that a stent, for example, can comprise a mesh, such as a metal mesh, that can be expanded in a narrowed vessel to prop the vessel open to allow blood to flow through. Often, the narrowing is caused by plaque that is deposited in the lumen, and the plaque can be rough or uneven, resulting in a rough lumen. Oftentimes, after the stent is expanded, the struts on the stent may not be able to conform to the pits or grooves on the lumen surface and result in a gap between lumen surface and the stent strut. This can be referred to as a “non apposed” or “malapposed” stent strut, for example. The gap from the malapposed strut, for example, can trap blood into a stagnant or unsteady state resulting in thrombus formation. Such a condition can easily occur with drug eluting stents (DES), because of lack of tissue healing over the strut, or surrounding luminal tissue eroding from release of the drug from the stent. Thrombus formation can occur, for example, and lead to rapid occlusion of the vessel. As such, the condition can cause major complications or death. Oftentimes, depressions or protrusion from an uneven calcified lumen surface can prevent struts from apposing even under high-pressure balloon inflation.

As such, the art would appreciate a method, system and device for focally pushing an intraluminal device against a luminal surface of a vessel to eliminate the gap created by a non-apposed, or malapposed, relationship between the intraluminal device and the luminal surface of the vessel.

### SUMMARY

The teachings provided herein are generally directed to a method, device and system for use in reducing, inhibiting, or correcting a malapposed intraluminal device. An example of such an intraluminal device is a stent and, in some embodiments, a cardiac stent. In some embodiments, for example, the method, device, and system includes an elongated device, such as an angioplasty balloon. The angioplasty balloon is designed with a special bump on the outer surface of the balloon to engage and focally push on a luminal surface of a non-apposed, or malapposed, stent strut. The pressure is applied focally, for example, to reduce, inhibit, or correct the malapposition of the abluminal surface of the stent strut with respect to an adjacent vessel wall of a vascular lumen, such as a coronary artery, in a subject.

The teachings include a device for reducing, inhibiting or correcting a malapposed intraluminal device. The device can comprise an expandable member for applying a focused region of pressure on a luminal surface of an intraluminal device, the luminal surface corresponding to a malapposed region between an abluminal surface of the device and a surface of the lumen of a subject. The expandable member can be positioned in the lumen of the subject, and expanded, for example, to apply the focused region of pressure to the luminal region of the intraluminal device to reduce, inhibit, or correct the malapposed intraluminal device.

In some embodiments, the expandable member can comprise an expandable casing having a protrusion that applies the focused region of pressure, wherein the expandable member can include a balloon, for example, the balloon having a protrusion that applies the focused region of pressure.

The teachings include a system for reducing, inhibiting or correcting a malapposed intraluminal device, the system comprising components. In some embodiments, the components include an intraluminal device; and a catheter comprising an expandable member. The expandable member can be in an unexpanded configuration and capable of being expanded to apply a focused region of pressure on a luminal surface of an intraluminal device. The luminal surface can correspond to a malapposed region between an abluminal surface of the device and a surface of the lumen of a subject.

The teachings include a medical assembly, the medical assembly comprising a stent and a catheter assembly. The catheter assembly can comprise a balloon having the stent positioned on the balloon, the balloon having a deflated configuration and capable of being enlarged to an expanded configuration to apply a focused region of pressure on a luminal surface of an intraluminal device, the luminal surface of the intraluminal device corresponding to a malapposed region between an abluminal surface of the stent and a surface of the lumen of a subject.

The teachings include method for constructing the medical assembly. The method includes placing the stent over the balloon of the catheter assembly, such that in response to the enlargement of the balloon, the focused region of pressure is applied to a luminal surface of the stent. The focused region of pressure corresponds to the malapposed region between the abluminal surface of the stent and the surface of the lumen of the subject.

The teachings include a method of reducing, inhibiting or correcting a malapposed intraluminal device. The method includes delivering an intraluminal device to a lumen of a subject using a system that includes the intraluminal device and a catheter comprising an expandable member. The expandable member can be in an unexpanded configuration prior to delivery and capable of being expanded to apply a focused region of pressure on a luminal surface of the intraluminal device. The luminal surface can correspond to a malapposed region between an abluminal surface of the device and a surface of the lumen of the subject. As such, the method can further include expanding the expandable member to apply the focused region of pressure to reduce, inhibit, or correct the malapposed intraluminal device.

The expandable member can comprise an expandable casing having a protrusion that applies the focused region of pressure. In some embodiments, the expandable member comprises a balloon having a protrusion that applies the focused region of pressure.

The protrusion can be any protrusion that one of skill will find useful in reducing, inhibiting, or correcting a malapposition between a region of an intraluminal device and a surface of a wall of a lumen of a subject. For example, the protrusion can comprise a top surface for contacting the luminal surface of the intraluminal device, the top surface having a shape that is convex, concave, round, flat, saddle shaped, or some combination thereof.

Moreover, the intraluminal device can be any device known to one of skill, the implementation of which might benefit from the teachings provided herein. In some embodiments, the intraluminal device is a stent. In some embodiments, the intraluminal device is a cardiac stent.

### BRIEF DESCRIPTION OF THE FIGURES

FIG. 1 illustrates a malapposing stent that is leaving a gap between a stent strut and a lumen surface of a subject, according to some embodiments.

FIGS. 2A and 2B illustrate the procedure of delivering an appositioner catheter and activating the mechanism to correct a malapposed stent strut, according to some embodiments.

FIG. 3 illustrates a method for forming an appositioner balloon, according to some embodiments.

FIGS. 4A and 4B illustrate a correction of a malapposed stent strut on an aortic valve deployed using a transcatheter aortic valve implantation (TAVI) approach, according to some embodiments.

FIG. 5 illustrates a malapposed stent strut in an aortic valve in a cadaver, according to some embodiments.

### DETAILED DESCRIPTION OF THE TEACHINGS

The teachings provided herein are generally directed to a method, device and system for use in reducing, inhibiting, or correcting a malapposed intraluminal device. An example of such an intraluminal device is a stent and, in some embodiments, the intraluminal device can be any device known to one of skill, the implementation of which might benefit from the teachings provided herein. In some embodiments, the intraluminal device is a cardiac stent.

One of skill will appreciate having a device for reducing, inhibiting or correcting a malapposed intraluminal device. The device can comprise an expandable member for applying a focused region of pressure on a luminal surface of an intraluminal device, the luminal surface corresponding to a malapposed region between an abluminal surface of the device and a surface of the lumen of a subject. The expandable member can be positioned in the lumen of the subject, and expanded, for example, to apply the focused region of pressure to the luminal region of the intraluminal device to reduce, inhibit, or correct the malapposed intraluminal device. The term “subject” and “patient” can be used interchangeably and refer to an animal such as a mammal including, but not limited to, non-primates such as, for example, a cow, pig, horse, cat, dog, rat and mouse; and primates such as, for example, a monkey or a human. As such, the terms “subject” and “patient” can also be applied to non-human biologic applications including, but not limited to, veterinary, companion animals, commercial livestock, and the like.

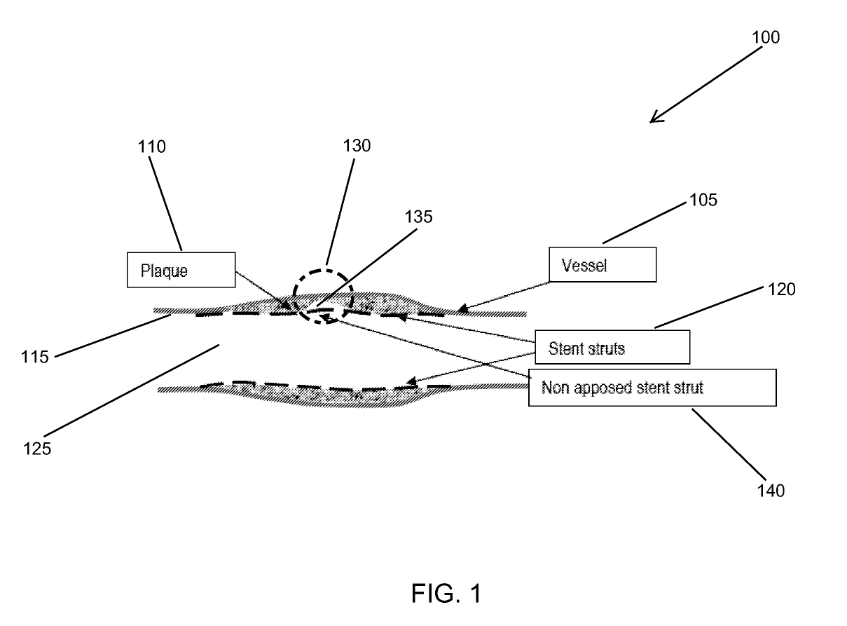

FIG. 1 illustrates a malapposing stent that is leaving a gap between a stent strut and a lumen surface of a subject, according to some embodiments. Treated region 100 includes a vessel 105 containing plaque 110 on a luminal surface 115 of the vessel 105. Stent 120 has been delivered and positioned within lumen 125. Region 130 forms a gap between an abluminal surface 135 of a malapposed stent strut 140 and the luminal surface 115 of the vessel 105.

Likewise, one of skill will appreciate having a system for reducing, inhibiting or correcting a malapposed intraluminal device. In some embodiments, the components of the system include an intraluminal device; and a catheter comprising an expandable member. The expandable member can be in an unexpanded configuration and capable of being expanded to apply a focused region of pressure on a luminal surface of an intraluminal device. The luminal surface can correspond to a malapposed region between an abluminal surface of the device and a surface of the lumen of a subject.

In some embodiments, the expandable member comprises an expandable casing having a protrusion that applies the focused region of pressure. And, in some embodiments, the expandable member comprises a balloon having a protrusion that applies the focused region of pressure.

The teachings include a medical assembly, the medical assembly comprising a stent and a catheter assembly. The catheter assembly can comprise a balloon having the stent positioned on the balloon, the balloon having a deflated configuration and capable of being enlarged to an expanded configuration to apply a focused region of pressure on a luminal surface of an intraluminal device, the luminal surface of the intraluminal device corresponding to a malapposed region between an abluminal surface of the stent and a surface of the lumen of a subject.

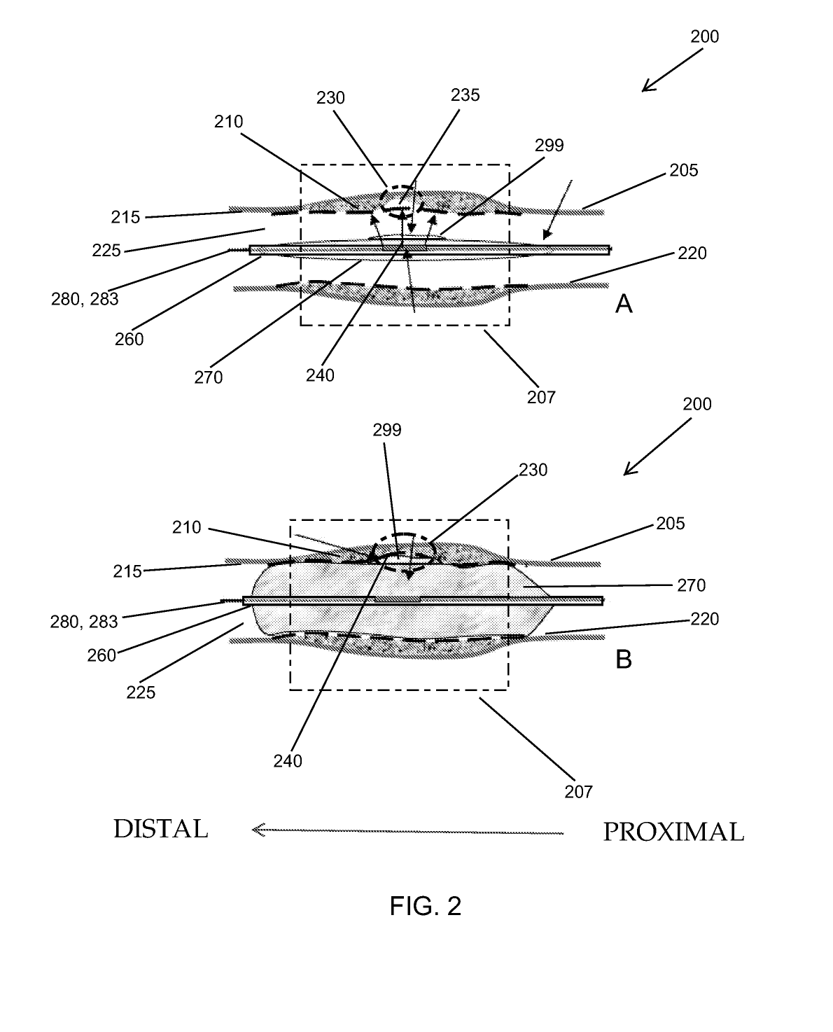

FIGS. 2A and 2B illustrate the procedure of delivering an appositioner catheter and activating the mechanism to correct a malapposed stent strut, according to some embodiments. Treated region 200 includes a vessel 205 containing plaque 210 on a luminal surface 215 of the vessel 205. Stent 220 has been delivered and positioned within lumen 225. Region 230 forms a gap between an abluminal surface 235 of a malapposed stent strut 240 and the luminal surface 215 of the vessel 205.

FIG. 2A shows a catheter 260 having a balloon 270 collapsed and folded. The catheter 260 can be advanced over the guidewire 280 until it passes through the lumen of the stent 220 deployed in a lesion 207 having plaque 210, until an occluder balloon (not shown) is distal to the lesion 207, at which time the occluder balloon can be inflated to occlude the flow of blood. The guidewire 280 can then be exchanged for an imaging wire 283 such as an optical coherence tomography wire (OCT). Saline can be used to flush-out blood from the imaging wire 283. The imaging wire 283 can then be pulled back to identify a malapposed stent strut 240. The catheter 260 can then be pulled back axially until a protrusion 299 on an appositioner balloon 270 can be lined up with the malapposed stent strut 240.

FIG. 2B shows a correction of the malapposed stent strut. The occluder balloon (not shown) can be deployed with inflation at low pressure using a pressure ranging from about 1 to about 3 atmospheres, for example. The catheter 260 can then be rotated so that the protrusion 299 is radially lined up with the malapposed stent strut 240. The appositioner balloon 270 can then be further inflated to push the protrusion 299 up to the malapposed stent strut 240 using a pressure ranging from about 4 to about 6 atmospheres, for example. Small adjustments in the position of the protrusion 299 can be made by translating the catheter 260 axially or rotating the catheter 260 to line up the protrusion 299 with the malapposed stent strut 240. The appositioner balloon 270 can be inflated, for example, using any suitable pressure or fluid known to one of skill. In some embodiments, the fluid can be a gas or a liquid. The appositioner balloon 270 can be inflated fully to push the malapposed stent strut 240 against the luminal surface 215 of the vessel 205 using a pressure ranging from about 7 to about 30 atmospheres, for example. The occluder balloon (not shown) and the appositioner balloon 270 can then each be deflated and repositioned with the imaging wire 283 to treat the next malapposed stent strut (not shown).

One of skill will appreciate having a method for constructing the medical assembly. It should be appreciated that any number and combination of manufacturing processes are known to one of skill in manufacturing such devices, and any one or combination of such processes can be used to implement the teachings provided herein. The method includes placing the stent over the balloon of the catheter assembly, such that in response to the enlargement of the balloon, the focused region of pressure is applied to a luminal surface of the stent. The focused region of pressure corresponds to the malapposed region between the abluminal surface of the stent and the surface of the lumen of the subject.

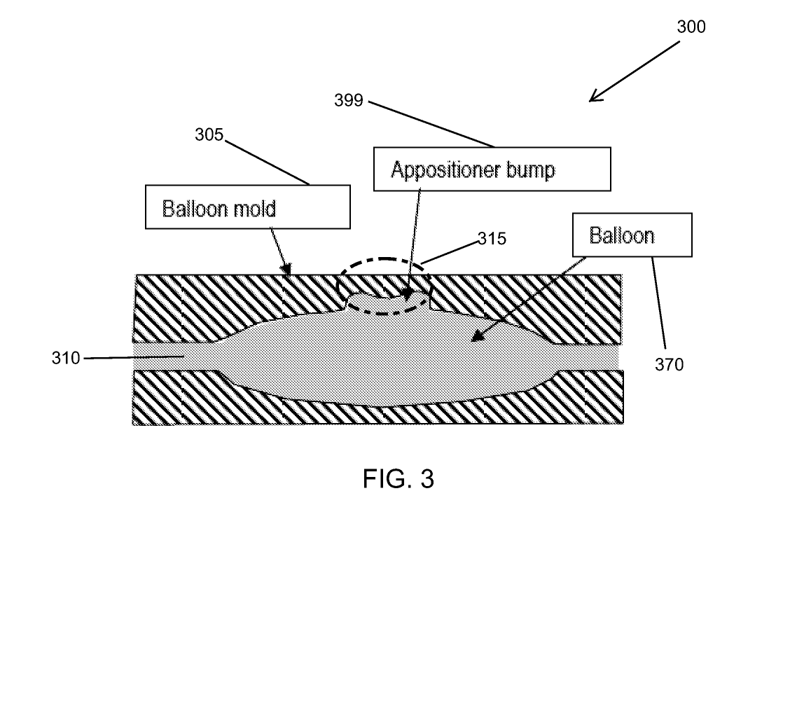

FIG. 3 illustrates a method for forming an appositioner balloon, according to some embodiments. System 300 includes a balloon mold 305, a balloon material 310, and a recess 315 in the balloon mold 305 to form a protrusion 399 of an appositioner balloon 370. The protrusion 399 can be shaped in any suitable dimension or geometry recognized by one of skill for the applications taught herein. In some embodiments, the protrusion 399 can have a top that is flat, convex, concave, or saddle shaped when fully deployed. The protrusion 399 can be directed to protrude radially outward, beyond a non-protruding surface of an inflated balloon, for example.

The balloon material can be any material known to one of skill to be useful for the teachings provided herein. Physical characteristics to consider may include elasticity, hardness, Young's modulus, coefficient of friction, surface tension, biocompatibility, and the like. One of skill will be able to design and select a suitable material for use in select target locations for deployment of the appositioner balloon. In some embodiments, the balloon material 310 can comprise an optically clear material. Examples of such materials can include, but are not limited to nylon, polyethylene, fluoropolymer, polyether block amide (PEBAX), polyurethane, silicone, polycarbonate or polyethylene terephthalate (PET).

A direct visualization component can be included in the teachings provided herein, such that the non-apposing, or malapposed, strut can be located, allowing a physician to direct the appositioner balloon against the luminal surface of the strut in the target area. The target area is the luminal surface of the strut that could receive a regionally focused pressure from the appositioner balloon to reduce, inhibit, or correct the positioning of the non-apposed, or malapposed, strut. Direct visualization means can include, for example, intravascular ultrasound, angioscopy, fiberoptics, and optical coherence tomography. And, the direct visualization means can be configured to allow the visualization means to slide axially relative to the device. Likewise, the protrusion can be opaque to visualization, such as, for example, a protrusion that is radioopaque. In some embodiments, the protrusion may be partially transparent to the direct visualization means. Any radioopaque material known to one of skill in the art to be biologically safe and useful for the teachings provided herein can be used. In some embodiments, the protrusion 399 can be filled with barium sulfate or bismuth, for example, and/or it can have a metal such as, for example, stainless steel, platinum, tungsten, iridium, or gold, embedded or attached to the top of the protrusion.

The protrusion can be any protrusion that one of skill will find useful in reducing, inhibiting, or correcting a malapposition between a region of an intraluminal device and a surface of a wall of a lumen of a subject. For example, the protrusion can comprise a top surface for contacting the luminal surface of the intraluminal device, the top surface having a shape that is convex, concave, round, flat, saddle shaped, or some combination thereof. The protrusion can include any shape, size, geometry, or configuration that one of skill may consider useful for the teachings provided herein. Any of a vast variety of shapes, dimensions, and geometries can be used, the selection of which can be subjective to a particular application, condition, or subject. In some embodiments, the protrusion can have a dimension ranging from about 0.002 inches to about 0.040″ in height, from about 0.003 inches to about 0.040 inches wide, and form about 0.003 inches to about 0.040 inches in length.

The expandable member can have any number of protrusions, each protrusion can be the same or different in composition, size, geometry, dimensions, hardness, elasticity, functionality and the like. Each protrusion can be independently selected for a particular function. And each protrusion can be located at any position on the surface of the expandable member, for example, such that the protrusion can be positioned to provide a force on a luminal surface of an intraluminal device. In some embodiments, a protrusion can be located near the midpoint between the ends of a balloon, for example.

The protrusion can be formed on an expandable member using any method known to one of skill in the art of manufacturing such devices. For example, the protrusion can be uninflated, or deflated, and expand as a bulge upon inflation of a balloon. In some embodiments, the protrusion can include a molded or machined piece bonded to the balloon material, in some embodiments. Any method of bonding known to one of skill can be used. In some embodiments, the bonding and bonded material is biocompatible or biologically safe for purposes of the teachings described herein. For example, a bonding process can include heat welding, gluing, snap fitting, or suturing using process known to one of skill as biologically safe and biocompatible to a subject. In some embodiments, the protrusion can be a separate component, separate from the balloon material, the separate component being added to the balloon material in the molding process. In some embodiments, the separate component can be molded into the balloon material. Moreover, the protrusion can also be part of the balloon material that is shaped such that the balloon bulges out more to the shape of the protrusion under pressure. In this case the balloon material can be a polymer tube, such as nylon 12, for example, placed in a mold that can comprise a glass, a metal, or an alloy.

The system 300 provides a final shape for an appositioner balloon, in which the balloon mold 305 includes a recess 315 in a position and shape of the protrusion 399. The balloon material 310 can then be pressurized with heat to expand to the shape of the balloon mold 305 and further heated to a temperature, for example, ranging from about 300 to about 500 degrees F. for a duration of time that can be easily selected by one of skill in the art of such manufacturing processes. One of skill can select a period of time that is known to be suitable for the balloon material 310 selected, for example. In some embodiments, the duration of time for the further heat can range from about 1 minute to about 20 minutes. In some embodiments, the balloon mold 305 can then be cooled while maintaining a pressure. In such embodiments, an appositioner balloon having a protrusion of a desired shape in a desired location on the appositioner balloon is the result.

The expandable member can comprise an expandable casing having a protrusion that applies the focused region of pressure. In some embodiments, the expandable member comprises a balloon having a protrusion that applies the focused region of pressure. In some embodiments, the expandable member can be comprised of tines that are expandable using a mechanical application of force, or a fluid pressure, for example.

The material for an expandable casing can include any material known to one of skill to be useful to the teachings provided herein. In some embodiments, the material can include nylon, polyethylene, PEBAX, PET, silicone, polyurethane or a combination thereof. In some embodiments, the material can comprise nylon or PEBAX.

The appositioner balloon can be designed to have any shape known to one of skill to be suitable for the teachings provided herein, to the extent, for example, that the shape reflects a region of an intraluminal device that may have the problem of malapposition after deployment in a subject. In some embodiments, the appositioner balloon can be oblong in shape, and of any size known to be suitable to one of skill for the chosen application. Chosen applications can include any application of a stent in any lumen of a subject. Such lumens can include any vascular lumen, a cardiac lumen, a lumen of a urinary vessel, a lumen of an intestinal vessel, a lumen of a respiratory vessel, and the like.

It should be appreciated that a stent can resemble a tube-like body that is used to open a lumen within an organ in a mammal, maintain lumen patency, or reduce the likelihood that the lumen will narrow again. Examples of such organs include, but are not limited to, vascular organs such as, for example, coronary arteries or hepatic veins; renal organs such as, for example, urethras and ureters; biliary organs such as, for example, biliary ducts; pulmonary organs such as, for example, tracheas, bronchi and bronchioles; and gastrointestinal organs such as, for example, esophagi and colons, to name a few.

The appositioner balloon can have a diameter in an uninflated, deflated, or inflated state, and the diameter in any given state can depend on the balloon selected, the use of the balloon, and the step in the process in which the balloon is being used. In some embodiments, the balloon can have, for example, an uninflated diameter ranging from about 0.5 mm to about 2 mm, from about 0.5 mm to about 4 mm, from about 0.5 mm to about 1 mm, from about 0.5 mm to about 3 mm, from about 0.5 mm to about 2 mm, from about 0.5 mm to about 2 mm, from about 0.5 mm to about 15 mm, from about 0.5 mm to about 12 mm, from about 0.5 mm to about 10 mm, from about 0.5 mm to about 8 mm, from about 0.5mm to about 5 mm or any range therein in increments of 0.1 mm.

Since the intraluminal device can be placed in a variety of organs within a mammal, it can have a variety of dimensions. In some embodiments, the diameter of an intraluminal device can range from about 0.025 mm to about 50 mm, from about 0.05 mm to about 25 mm, from about 0.1 mm to about 20 mm, from about 0.25 mm to about 15 mm, from about 0.50 mm to about 10 mm, from about 1.0 mm to about 5 mm, or any range therein. In other embodiments, the diameter of an intraluminal device can range from about 0.05 mm to about 2.5 mm, from about 0.10 mm to about 2.0 mm, from about 0.25 mm to about 1.5 mm, from about 0.50 mm to about 1.0 mm, or any range therein. In other embodiments, the diameter of an intraluminal device can range from about 10 mm to about 25 mm, from about 12 mm to about 22 mm, from about 15 mm to about 20 mm, or any range therein. In some embodiments, the length of an intraluminal device can range from about 0.1 mm to about 100 mm, from about 0.5 mm to about 75 mm, from about 1.0 mm to about 50 mm, from about 1.5 mm to about 40 mm, from about 2.0 mm to about 30 mm, or any range therein. The diameter of a catheter body can vary in correlation with the diameter of a stent, for example. The catheter body can be any length known to one of skill in the art to be useful with the teachings provided herein.

The intraluminal device can be composed of any materials and have any dimensions known to be useful to one of skill in the art. In some embodiments, the intraluminal device can include, but is not limited to, tubular stents, self-expanding stents, coil stents, ring stents, multi-design stents, and the like. In other embodiments, the intraluminal device can be metallic; low-ferromagnetic; non-ferromagnetic; biostable polymeric; biodegradable polymeric or biodegradable metallic. In some embodiments, the intraluminal device can be coated such as, for example, with a polymer containing a drug. The polymer can be exclusively on the outer surface, exclusively on the inner surface, or on both the outer surface and the inner surface of the intraluminal device.

In some embodiments, the intraluminal device can be composed of a metal, an alloy, a polymer, or a combination thereof. Examples of materials used to form stents include, but are not limited to, ELASTINITE (Guidant Corp.), NITINOL (Nitinol Devices and Components), stainless steel, tantalum, tantalum-based alloys, nickel-titanium alloy, platinum, platinum-based alloys such as, for example, platinum-iridium alloys, iridium, gold, magnesium, titanium, titanium-based alloys, zirconium-based alloys, alloys comprising cobalt and chromium (ELGILOY, Elgiloy Specialty Metals, Inc.; MP35N and MP20N (SPS Technologies), and combinations thereof. The tradenames “MP35N” and “MP20N” describe alloys of cobalt, nickel, chromium and molybdenum. The MP35N consists of 35% cobalt, 35% nickel, 20% chromium, and 10% molybdenum. The MP2ON consists of 50% cobalt, 20% nickel, 20% chromium, and 10% molybdenum.

The catheter body and the balloon can be made from any materials, and have any dimensions, known to be useful to one of skill in the art. Examples of materials used to make the catheter body can include, but are not limited to, polyvinyl chloride (PVC), polyethylene, silicone rubber, polyurethane, and any analogs, homologues, congeners, derivatives, salts, copolymers, and mixtures thereof. Examples of materials used to make the balloon can include, but are not limited to, latex, polyamide, nylon, polyethylene, low-density polyethylene (LDPE), DURALYN (Cordis Corp.), DURAMAX (Cordis Corp.), and any analogs, homologues, congeners, derivatives, salts, copolymers, and mixtures thereof.

In some embodiments, the appositioner balloon can have inflated diameter ranging from about 2 mm to about 10 mm, and a length ranging from about 3 mm to about 8 mm. In some embodiments, the appositioner balloon can range from about 3 to about 17 mm in length and from about 2 to about 35 mm in diameter, depending on the vessel diameter fully inflated. For coronary arteries, for example, the appositioner balloon can range from about 2 to about 4 mm in diameter and from about 3 to about 6 mm in length. For aorta or valves, for example, the appositioner balloon can range from about 4 to about 17 mm in length and from about 20 to about 30 mm in diameter. The appositioner balloon can be tapered on the proximal and distal end, for example, to provide a surface for bonding to a delivery catheter. Moreover, the balloon material can have any thickness known to one of skill to be useful to the teachings provided herein. In some embodiments, the appositioner balloon can range from about 0.0005 to about 0.008″ in thickness, from about 0.001 to about 0.005 inches in thickness, or any range therein.

The catheter shaft can be a tube having any diameter considered useful and applicable to one of skill in the art, as applied to the teachings herein. In some embodiments, the catheter shaft can be a tube with outer diameter ranging from about 0.5 mm to about 8 mm, from about 0.5 mm to about 2 mm, from about 0.5 mm to about 5 mm, from about 0.5 mm to about 1 mm, or any range therein. The catheter shaft can have a balloon inflation port near the tip, as well as a flush port configured for delivering fluid from outside body to space around balloon. The catheter can include an inner tube within an outer tube, the inner tube having, for example, an inner diameter ranging from about 0.014 inches to about 0.050 inches. Likewise, it should be appreciate that catheters can be designed to have any of a variety of lengths, depending on the use, and any such length catheter can be designed for use with the teachings herein. In some embodiments, the catheter can range in length, for example, from about 90 cm to about 150 cm. The catheter can accommodate a guidewire in some embodiments. In some embodiments, the catheter can be optically passable, translucent or transparent, for example, at the distal end, or tip, of the catheter to allow an imaging device to operate through the catheter. The shaft can have a metal braid (stainless steel, for example) or an embedded metal coil, such as one or more helical coils wound around the catheter, to provide a torsional strength and stiffness, while remaining flexible for bending, but stiff in rotation or in receiving an axial force, to assist in traversing through a tortuous vessel, such as a tortuous vasculature, for example.

In some embodiments, the catheter can have an occluder balloon, such as a proximal balloon, that is positioned to occlude blood, for example. In some embodiments, the catheter includes a flush port to flush away blood that is distal to the proximal balloon, for example, to clear out blood for visualization using methods such as OCT.

Other ways to effect apposition can include having an arm that protrude out of a side port of the catheter, the arm functioning to push on the strut. In some embodiments, a basket having a series of longitudinal tines, wires, bands, or the like, can be introduce to the lumen of a vessel and expanded to reduce, inhibit, or correct a non-apposed, or malapposed, intraluminal device. A tine, for example, can have a protrusion on the outer most point of an arch of the tine. In some embodiments, a catheter can also be designed to flex by pulling on a side tendon of the catheter to point the tip towards the a non-apposed, or malapposed, region of an intraluminal device, to create a regionally focused pressure on the luminal surface of the device to reduce, inhibit, or correct the relation of the device to the vessel wall.

As such the teachings provided herein include a method of reducing, inhibiting or correcting a malapposed intraluminal device. The method includes delivering an intraluminal device to a lumen of a subject using a system that includes the intraluminal device and a catheter comprising an expandable member. The expandable member can be in an unexpanded configuration prior to delivery and capable of being expanded to apply a focused region of pressure on a luminal surface of the intraluminal device. The luminal surface can correspond to a malapposed region between an abluminal surface of the device and a surface of the lumen of the subject. As such, the method can further include expanding the expandable member to apply the focused region of pressure to reduce, inhibit, or correct the malapposed intraluminal device.

One of skill will appreciate that stents can serve several functions. For example, stents that act as a frame for percutaneous valves in transcatheter aortic valve implantation (TAVI), a procedure to treat patients with severe aortic stenosis and high or prohibitive risk for standard surgical management, are often malapposed and can lead to paravalvular leakage. An increased chance of migration, poor blood flow, or blood clotting can also occur.

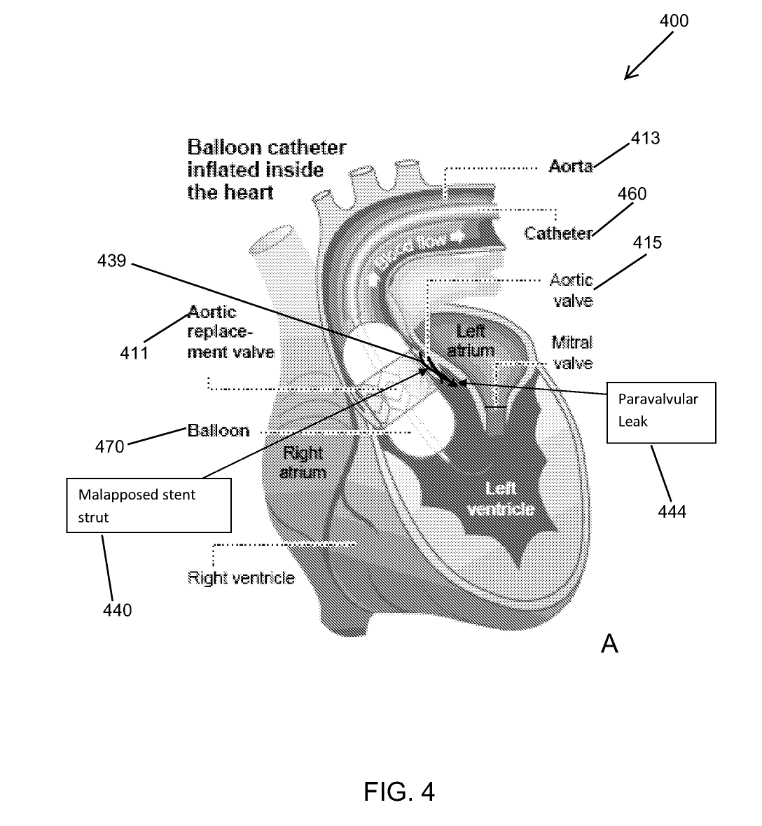

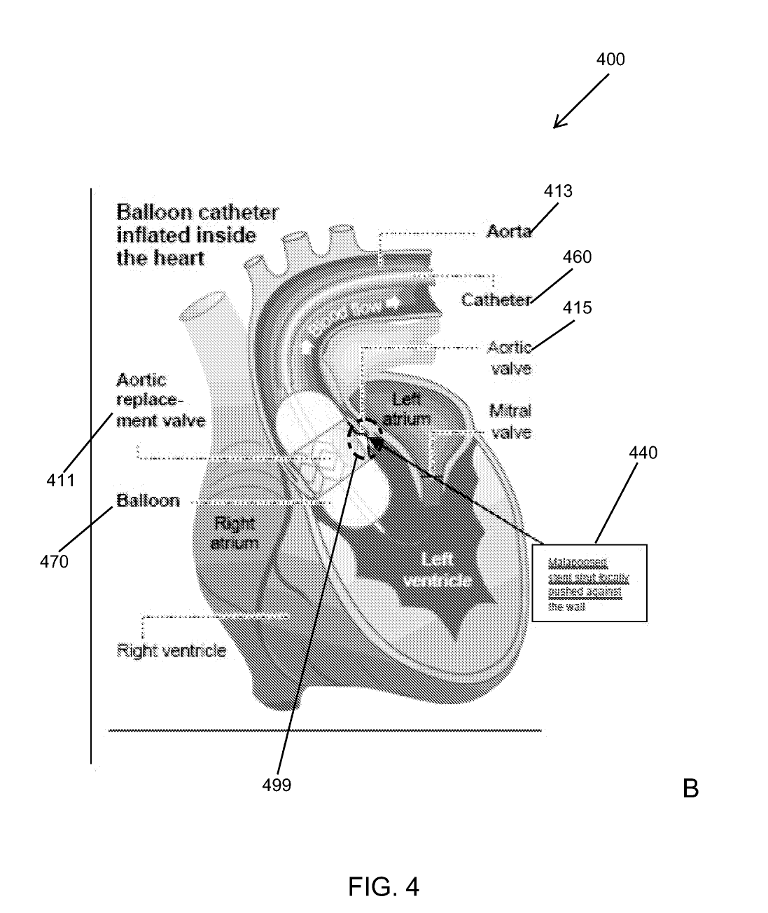

FIGS. 4A and 4B illustrate a correction of a malapposed stent strut on an aortic valve deployed using a transcatheter aortic valve implantation (TAVI) approach, according to some embodiments. In FIG. 4A, a treated heart 400 contains an aortic replacement valve 411 in aorta 413 to open aortic valve 415. Catheter 460 with an appositioner balloon 470 can be delivered through the aorta 413 to apply a focused regional pressure to a luminal surface 439 of a malapposed stent strut 440 to correct a paravalvular leak 444. FIG. 4B shows the use of a protrusion 499 used to correct the malapposition that created the paravalvular leak 444, for example, between the aortic valve 415 and the malapposed stent stent 440.

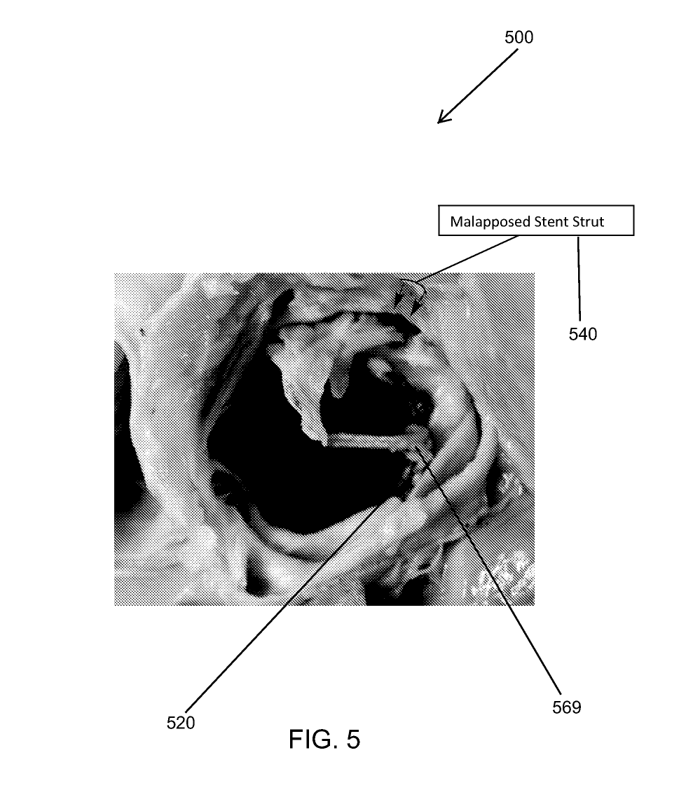

FIG. 5 illustrates a malapposed stent strut in an aortic valve in a cadaver, according to some embodiments. Aortic region 500 contains aortic valve 569 containing a stent 520 having a malapposed stent strut 540.

While various exemplary embodiments have been described, those skilled in the art will realize that there are many alterations, modifications, permutations, additions, combinations, and equivalents which fall within the true spirit and scope of the teachings. It is therefore intended that the preceding descriptions not be read by way of limitation but, rather, as examples with the broader scope of the concepts disclosed herein.

We claim:

1. A device for reducing, inhibiting or correcting a malapposed intraluminal device, comprising:
an expandable member for applying a focused region of pressure on a luminal surface of an intraluminal device that corresponds to a malapposed region between an abluminal surface of the device and a surface of the lumen of a subject;
wherein, the expandable member can be positioned in the lumen of the subject, and expanded, to apply the focused region of pressure to the luminal region of the intraluminal device to reduce, inhibit, or correct the malapposed intraluminal device.

2. The device of claim 1, wherein the expandable member comprises an expandable casing having a protrusion that applies the focused region of pressure.

3. The device of claim 1, wherein the expandable member comprises a balloon having a protrusion that applies the focused region of pressure.

4. The device of claim 1, wherein the intraluminal device is a stent.

5. The device of claim 1, wherein the intraluminal device is a cardiac stent.

6. A system for reducing, inhibiting or correcting a malapposed intraluminal device, comprising:
an intraluminal device;
a catheter comprising an expandable member, the expandable member in an unexpanded configuration and capable of being expanded to apply a focused region of pressure on a luminal surface of an intraluminal device, focused region of pressure corresponding to a non-apposed, or malapposed, region between an abluminal surface of the device and a surface of the lumen of a subject.

7. The system of claim 6, wherein the expandable member comprises an expandable casing having a protrusion that applies the focused region of pressure.

8. The system of claim 6, wherein the expandable member comprises a balloon having a protrusion that applies the focused region of pressure.

9. The system of claim 6, wherein the intraluminal device is a stent.

10. The system of claim 6, wherein the intraluminal device is a cardiac stent.

11. A medical assembly, comprising:
a stent;
a catheter assembly comprising a balloon having the stent positioned on the balloon, the balloon having a deflated configuration and capable of being enlarged to an expanded configuration to apply a focused region of pressure on a luminal surface of an intraluminal device, the focused region of pressure corresponding to a non-apposed, or malapposed, region between an abluminal surface of the stent and a surface of the lumen of a subject.

12. The assembly of claim 11, wherein the stent is a cardiac stent.

13. A method for constructing the medical assembly of claim 11, comprising:
placing the stent over the balloon of the catheter assembly, such that in response to the enlargement of the balloon, the focused region of pressure is applied to a luminal surface of the stent, the focused region of pressure corresponding to a non-apposed, or malapposed, region between the abluminal surface of the stent and the surface of the lumen of the subject.

14. The method of claim 13, wherein the stent is a cardiac stent.

15. A method of reducing, inhibiting or correcting a malapposed intraluminal device, comprising:
using the system of claim 6 to deliver the intraluminal device to the lumen of the subject; and,
expanding the expandable member to apply the focused region of pressure to reduce, inhibit, or correct the malapposed intraluminal device.

16. The method of claim 15, wherein the expandable member comprises an expandable casing having a protrusion that applies the focused region of pressure.

17. The method of claim 15, wherein the expandable member comprises a balloon having a protrusion that applies the focused region of pressure.

18. The method of claim 15, wherein the intraluminal device is a stent.

19. The method of claim 15, wherein the intraluminal device is a cardiac stent.

20. The method of claim 15, wherein the protrusion comprises a top surface for contacting the luminal surface of the intraluminal device, the top having a shape that is convex, concave, round, flat, or saddle shaped.
